(19) United States
(12) Patent Application Publication
(10) Pub. No.: US 2002/0110840 A1
(43) Pub. Date: Aug. 15, 2002

(54) Screening method for identifying compounds that selectively induce interferon alpha

(75) Inventors: Mark A. Tomai, Woodbury, MN (US); John P. Vasilakos, Woodbury, MN (US)

Correspondence Address: MarySusan Howard, Office of Intellectual Property Counsel, 3M Innovative Properties Company, P.O. Box 33427, St. Paul, MN, 55133-3427, US

(73) Assignee: 3M Innovative Properties Company

(21) Appl. No.: 10/013,193

(22) Filed: Dec. 6, 2001

Related U.S. Application Data
(60) Provisional application No. 60/254,229, filed on Dec. 8, 2000.

(51) Int. Cl. G01N 33/567; G01N 33/53; G01N 33/537; G01N 33/543

(52) U.S. Cl. 435/7.21; 435/7.92

(57) ABSTRACT

Methods for screening for compounds that selectively induce IFN-&60 production and methods for ameliorating conditions in a patient using a small molecule that selectively induces the production of IFN-α are disclosed.

This application claims the benefit of previously filed Provisional Application Serial No. 60/254,229, filed on Dec. 8, 2000.

### FIELD OF THE INVENTION

The present invention is directed to the field of immunology and specifically to modulation of the immune response. The invention provides methods of screening compounds for selective induction of IFN-α and compounds and methods for selective induction of IFN-α to ameliorate conditions in patients which are responsive to IFN-α.

### BACKGROUND

Interferon-α (IFN-α) can be used to treat a variety of conditions. For example, IFN-α can be used to treat conditions such as hepatitis, multiple sclerosis, various dermatological disorders associated with hepatitis C, lymphoma and melanoma. Recently, it was shown that the primary interferon-producing cell in the blood in response to viral infections is the plasmacytoid dendritic cell (pDC). These cells are required for and are the principal IFN-producing cells in the blood in response to the class of compounds known as the imidazoquinolines. Such compounds are disclosed in, for example, U.S. Pat. No. 4,689,338; 5,389,640; 5,268,376; 4,929,624; 5,266,575; 5,352,784; 5,494,916; 5,482,936; 5,346,905; 5,395,937; 5,238,944; 5,525,612; 5,175,296; 5,693,811; 5,741,908; 5,939,090; 6,110,929; 4,988,815; 5,376,076; and PCT Publications WO 99/29693; WO 00/76505; WO 00/76518; and WO 00/76519. All these applications are incorporated herein by reference.

However, immune responses, or compounds that induce immune responses, such of the production of IFN-α can also upregulate the production of inflammatory cytokines such as Tumor Necrosis Factor-α (TNF-α) and IL-1. Upregulation of inflammatory cytokines such as TNF-α and IL-1 can often have detrimental effects, such as tissue destruction. In many clinical situations it may be desirable to limit the production of inflammatory cytokines while still inducing the production of IFN-α. Accordingly, there is a need to develop compounds and methods to screen for compounds that induce IFN-α production without significantly increasing the production of inflammatory cytokines.

### SUMMARY OF THE INVENTION

The present invention provides a method for identifying compounds that stimulate the production of IFN-α without concomitant production of significant levels of inflammatory cytokines, such as TNF-α, from cells present in the bloodstream. The method involves screening potential compounds on a population of cells that contain pDC2 cells, which are responsible for the majority of the production of the IFN-α in the population. Compounds that meet this criteria are designated as “selective compounds”. The invention also provides a method for using a selective compound of the invention to affect a condition in a patient responsive to induction of IFN-α.

### DETAILED DESCRIPTION OF THE INVENTION

The present invention provides methods of identifying compounds that selectively induce production of IFN-α from pDC2 cells. In some embodiments, the invention provides for selective induction of IFN-α in a population of cells, such as unseparated whole blood or peripheral blood mononuclear cells (PBMC), without concomitant production of significant amounts of inflammatory cytokines such as TNF-α, IL-1, IL-6,, IL-8, IL-12, MCP-1, etc. The invention also provides preferred compounds for selective induction of IFN-α as well as compounds and methods for affecting a condition in a patient that is responsive to IFN-α. Administration of a compound that selectively induces IFN-α expression without expression of significant levels of inflammatory cytokines advantageously provides for targeted therapeutic or prophylactic effect with reduced likelihood of potentially undesired side affects from inflammatory cytokines.

As used herein, “pDC2 cell” means “precursor dendritic cell-type 2”, which is a plasmacystoid cell type that lacks leukocyte lineage markers, expresses CD4, MHC class II molecules, and CD123, and differentiates into type 2 dendritic cells when cultured with interleukin-3 with or without CD40 ligand. pDC2 cells can be identified by the presence of surface markers CD123+, HLA-DR+, CD4+, and the absence of leukocyte lineage specific markers and CD11C−. The term “pDC2 cell” is inclusive of these precursor cells and the differentiated type 2 dendritic cells (DC2).

The term “peripheral blood mononuclear cells” (PBMC) refers to cells that are typically found in blood and include T lymphocytes, B lymphocytes, NK cells, monocytes such as macrophages, and dendritic cells.

The term “inflammatory cytokine producing cells” includes a single cell type or combination of cell types that produce a major portion of the inflammatory cytokines within a population of PBMCs. Examples of such cells include monocytes, macrophages and dendritic cells that are CD11c+; (DC1) dendritic cells that are CD11c− are not considered to be “inflammatory cytokine producing cells” as that term is used herein.

The term “expression”, “expressed”, “expressing”, etc. refers to the production of a protein, and the messenger RNA (mRNA) that encodes the protein, from a gene.

An “inflammatory cytokine” refers to cytokines that induce an inflammatory response. Examples of inflammatory cytokines according to the invention include tumor necrosis factor-α (TNF-α), interleukin-1 (IL-1), IL-6, IL-8, and IL-12. As used herein, the term “inflammatory cytokine” does not include interferon α (IFN-α).

The term “pDC2-enriched cells” refers to a preparation of cells, for example PBMC, or whole blood cells, where the percentage of pDC2 cells is 5% or greater, preferably 20% or greater, more preferably 80% to 95%.

As used herein a “selective compound” refers to a compound that preferentially induces expression of IFN-α in a population of hematopoietic cells such as PBMCs containing pDC2 cells without concomitant production of signficant levels of inflammatory cytokines. As used in this context, the term “significant levels” refers to levels of inflammatory cytokines that cause an undesired effect by the inflammatory cytokines sufficient to reduce the utility of the compound for a particular application. For example, if the ratio of TNF-α produced to IFN-α produced is greater than about 1:3, this would be considered production of significant levels of an inflammatory cytokine.

### METHOD OF SCREENING

In one embodiment, the invention provides a method of identifying a compound that selectively induces production of IFN-α that includes screening the compound to determine whether the compound induces IFN-α production in a population of cells without significant induction of production of inflammatory cytokines, including TNF-α. A population of cells suitable for screening a compound according to the invention includes cells that produce inflammatory cytokines and pDC2 cells. Thus, examples of suitable cell populations include whole blood cells, complete or partial populations of PBMCs, PBMC cells enriched with pDC2 cell fraction, or any hematopoietic population containing pDC2 or DC2 cells.

In a typical embodiment, a selective compound of the invention induces expression of IFN-α predominantly from pDC2 cells. Preferably, a selective compound that induces IFN-α production does not induce high levels of inflammatory cytokines from pDC2 cells or other cells in the population of cells. In general, when a selective compound of the invention is administered to a population of cells including pDC2 cells and inflammatory cytokine producing cells, IFN-α is present in the population of cells in an amount at least three times greater than the amount of TNF-α, typically about 100 times greater, and in some embodiments about 1000 times greater or more.

A population of cells containing pDC2 cells can be obtained or prepared using any suitable method. For example, a blood cell sample for preparing a suitable population of cells can be obtained from most mammals. A cell sample may also be a population of cells subjected to enrichment or purification procedures to increase the percentage of a desired cell type, such as pDC2 cells, in the population of cells. These procedures can be based on either positive selection or negative selection. An example of positive selection is the process where a desired cell type is labeled with an antibody specific for that cell type, bound to a column where the binding is dependent on the presence of the antibody on that cell type, and then separated from other cells in the population. An example of negative selection is the process where undesired cells are labeled with antibodies directed against those cells, bound to a column where the binding is dependent on the presence of the antibodies, and then separated from the desired cell type. Such columns include, but are not limited to, for example, immunoaffinity-based columns or magnetic bead-based columns such as Dzionek A, Fuchs A, Schmidt P, Cremer S, Zysk M, Miltenyi S, Buck DW, Schmitz J: BDCA-2, BDCA-3, and BDCA-4: three markers for distinct subsets of dendritic cells in human peripheral blood. Journal of Immunology 165(11):6037, 2000.

Cells can also be enriched by positive or negative selection by sorting using a flow cytometer. According to this procedure, cells can be labeled with fluorophore-coupled antibodies to discriminate cell types and separated into populations based on the presence of the fluorophore-coupled antibodies on the cell surface. Other techniques for enriching cell populations for a desired cell type include, for example, ammonium lysis, complement cell lysis, density gradient separation, panning, adherence depletion, and charge-flow separation. It will be appreciated that these techniques may be performed alone, or in combination, to achieve the desired cell enrichment or purity.

In one embodiment of the invention, the population of cells containing pDC2 cells is contacted with a compound to be screened in an amount sufficient to assess the ability of the compound to selectively induce IFN-α expression. The culture conditions and composition of the media in which the population of cells are contacted with the compound can be performed using any suitable system. The specific amount of compound used to induce expression can vary, but stimulation typically is dose responsive. A typical dosage range for selective compounds is from about 0.005 to about 5 μM. However, compounds that are more potent stimulators of cytokine expression may display IFN-α production or inflammatory cytokine expression in a lower dosage range. Compounds can be applied to the cells in a suitable carrier that is physiologically compatible with the cells and the culture media.

The cell population is contacted with the compounds for a period of time sufficient to assess the ability of the compound to selectively induce IFN-α expression. The kinetics of expression of various cytokines is known in the art. However, the method of determining cytokine expression can also influence the time that the cell population is stimulated with a compound. For example, if cytokine expression is determined using a nucleic acid probe to assess the amount of intracellular cytokine mRNA produced, cells may generally be stimulated for a shorter period of time than when cytokine expression is determined by the amount of extracellular cytokine protein secreted into the media. Expression of intracellular mRNA or intracellular protein can in many cases be determined at about 2 to 6 hours after contacting the cell population with the compound. However, intracellular expression of IFN-α and TNF-α are typically determined from 6 to 24 hours after stimulation. Expression of extracellular protein can be determined at about 6 to 48 hours after contacting the cell population with the compound, typically about 12 to 36 hours.

The invention also provides for measurement of specific amounts or relative amounts of cytokines expressed from a cell population that has been contacted with a compound for a period of time. Thus, measurement of cytokines can be determined by assessing the amount of cytokine produced in the cell, for example, by immunodetection, utilizing multi-color flow cytometry. In one embodiment, antibodies against cytokines, such as IFN-α and TNF-α, can be used to penetrate a population of cells prepared using known techniques suitable for intracellular immunodetection. These antibodies can be coupled to compounds such as fluorophores, for example FITC, phycoerythrin and Cy3, or other fluorescent labels, which allow for the fluorescence detection of these antibodies and thus the relative amount of cytokine in the cells.

A flow cytometer can be used to measure the fluorescence in the cell population that has been stained with anti-IFN-α or anti-TNF-α fluorophore coupled antibodies. Optionally, a cell population can also be stained with fluorophore-coupled antibodies against specific surface proteins that allow for the discrimination of distinct cell types, for example, pDC2 cells, in the cell population. Identification of a desired cell type and the relative amount of cytokine expressed in that cell type, such as, the amount of IFN-α produced in pDC2 cells can be determined using multi-color flow cytometry.

Measurement of cytokine induction by immunodetection can also be determined by analyzing the amount of cytokine present extracellularly, or the amount of cytokine that has been secreted from the cell population into the culture media. Measurements of secreted IFN-α and TNF-α, for example, can be made using techniques such as ELISA or bioassay. Cytokines present in culture supernatants that were secreted from the cell population after stimulation with a compound for a period of time can be immobilized on plastic surfaces, such as microtiter plate surfaces. Antibodies, such as anti-IFN-α or anti-TNF-α antibodies, can be used to detect the presence of these cytokines immobilized on the plastic surface. These antibodies can be coupled to known detection moieties such as alkaline phosphatase or peroxidase molecules that can be used with a detection reagent to indicate the presence and relative amount of the cytokine. Cytokine standards can be run in parallel to determine the total amount of cytokine secreted into the media.

Measurement of cytokines by immunodetection or bioassay can also be determined by assessing the amount of cytokine present in cellular lysates. According to the invention, stimulated cells can be lysed or solubilized by known methods, for example, detergent lysis, and the lysates, which contain the cytokines, can be transferred to a support surface, for example a nitrocellulose membrane. Optionally, electorphoresis can be performed prior to transfer, to separate the constituents of the lysate. Western or dot blotting can be performed, utilizing appropriate secondary and detection reagents, to determine the presence and the amount of a cytokine, for example, IFN-α or TNF-α, in the cell lysate. Techniques for protein detection from cell lysates are commonly known in the art and can be found, for example, in Current Protocols in Protein Science (Ed.: Coligan et al., 1996, John Wiley & Sons, New York, N.Y.).

Methods for intracellular detection of cytokine expression also include detection of the amount of mRNA encoding a particular cytokine. According to this method, the amount of cytokine can be determined using a nucleic acid probe complimentary to the target cytokine mRNA, or a portion of the mRNA, in combination with flow cytometry and immunodetection, as described above. The nucleic acid probe can be coupled to a fluorophore, such as FITC or Cy3. Alternatively, cells stimulated with a particular compound can be harvested and lysed to generate lysates containing cytokine mRNA. Methods such as Northern analysis, for example, can be performed. According to one embodiment, Northern analysis can involve separating RNA by electrophoresis, transferring the RNA to a solid support, such as a membrane analysis, probing with a nucleic acid complimentary to the target cytokine mRNA and coupling to a detection moiety. Suitable detection moieties include a radiolabel, a fluorophore, a luminescent-generating compound, or a colorimetric compound. Other methods such as RNase protection assay (RPA) and RT-PCR can be used to determine the presence and amount of a cytokine mRNA, such as IFN-α or TNF-α mRNA, in a cellular sample. Methods for such assays are known and disclosed in, for example, Current Protocols in Molecular Biology (Ed.: Ausubel et al., 1990, Greene Pub. Associates and Wiley-Interscience: John Wiley, New York), which is incorporated herein by reference in its entirety.

### METHODS FOR AFFECTING A CONDITION IN A PATIENT

In another embodiment, the invention provides a method for affecting a condition of a patient responsive to IFN-α by administering a selective compound to the patient. The patient can be a human or animal. The selective compound provides an increase of IFN-α in a patient by increasing the expression of IFN-α from the patient's pDC2 cells. As discussed, the selective compounds preferably do not cause a significant increase in the expression of inflammatory cytokines.

Non-limiting examples of conditions which can be affected by increasing the level of IFN-α include melanoma, myeloid leukemia, non-Hodgkin's lymphoma, renal cell carcinoma, Kaposi's sarcoma, multiple sclerosis, hypereosinophilic syndrome, adenovirus, rhinovirus, variola (particularly variola major), influenza, coronavirus, HIV, para-influenza, myoproliferative disorders such as polycythemia vera, and idiopathic myelofibrosis, hepatitis B, chronic hepatitis C, and dermatological diseases such as cutaneous necrotising vasiculitis, mixed cryoglobulinemia, porphyria cutanea tarda, lichen planus, Adamantiadis-Behcet syndrom, erythema multiforme and nodosum, malacoplakia, urticaria pruritus, basal cell carcinoma, genital warts, actinic keratosis and other types of human papilloma virus infection including epidermoplasia verucciformis, common and plantar warts, and cervical dysplasia.

### SELECTIVE COMPOUNDS

Selective compounds include certain imidazoquinoline amines, imidazoquinoline sulfonamides, and imidazoquinoline ureas that increase the expression of IFN-α primarily from the pDC2 cells without significant expression of inflammatory cytokines.

Examples of compounds that have been identified as selective using the method of the invention include:

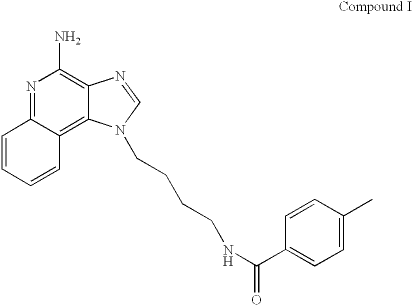

N-[4-(4-amino-1H-imidazo[4,5-c]quinolin-1-yl)butyl]-4-methylbenzamide;

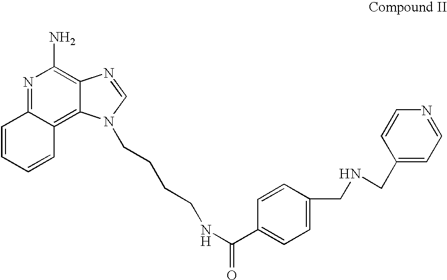

N-[4-(4-amino-1H-imidazo[4,5-c]quinolin-1-yl)butyl]-4-{[(pyridin-4-ylmethyl)amino]methyl}benzamide;

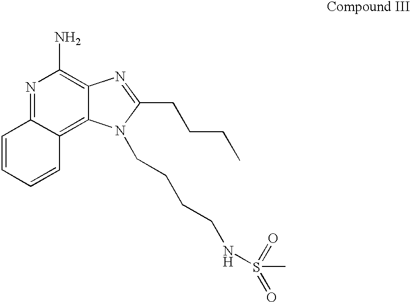

N-[4-(4-amino-2-butyl-1H-imidazo[4,5-c]quinolin-1-yl)butyl]methanesulfonamide;

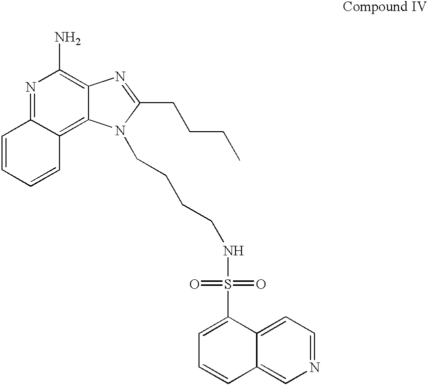

N-[4-(4-amino-2-butyl-1H-imidazo[4,5-c]quinolin-1-yl)butyl]isoquinoline-5-sulfonamide;

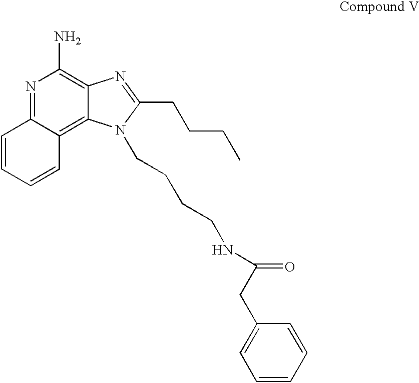

N-[4-(4-amino-2-butyl-1H-imidazo[4,5-c]quinolin-1-yl)butyl]-N′-phenylurea

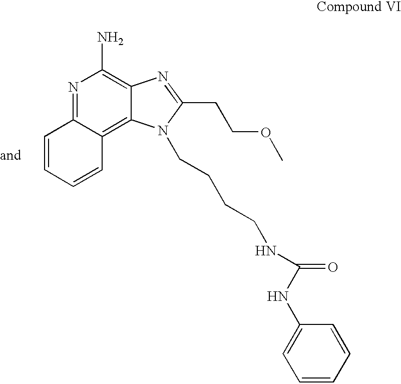

N-{4-[4-amino-2-(2-methoxyethyl)-6,7,8,9-tetrahydro-1H-imidazo[4,5-c]quinolin-1-yl]butyl}-N′-phenylurea

These compounds and methods of preparing them are described in more detail in WO 0076505; WO 0076518; and WO 0076519, the disclosures of which are incorporated by reference herein.

The compounds can also be targeted for specific delivery to a cell type to be treated by conjugation of the compound to a targeting moiety. Targeting moieties useful for conjugation to a compound such as an imidazoquinoline-based compound include antibodies, cytokines, and receptor ligands that are specific to the cell, in particular, pDC2 cells, to be affected. Targeting moieties for pDC2 cells can include, for example, anti-BDCA-2, anti-BDCA-4,anti-CD4 antibodies, anti-CD123 antibodies, or anti-HLA-DR antibodies.

If the selective compound of the invention is sufficiently basic or acidic to form stable nontoxic acid or base salts, administration of the compound as a salt may be appropriate. Examples of pharmaceutically acceptable salts are organic acid addition salts formed with acids which form a physiologically acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts. Pharmaceutically acceptable salts may be obtained using standard procedures known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The following examples are provided to further explain the invention through specific description of some embodiments of the invention. The Examples, however, are not intended to limit the scope of the invention.

### EXAMPLE

The following Example sets forth methods for screening IRM compounds which selectively induce cytokine production from pDC2 cells.

### Culture Medium

Complete RPMI (cRPMI) medium was used for the studies of these Examples. cRPMI was prepared by mixing RPMI 1640 with 25 mM HEPES (Life Technologies, Gaithersburg, Md.) supplemented with 10% heat inactivated (FCS) (Hyclone, Logan, Utah), 1 mM sodium pyruvate, 0.1 mM non-essential amino acids, 1 mM L-glutamine and 50 μg/ml gentamicin sulphate (Life Technologies).

### Generation, Purification or Enrichment of Various Cell Types

PBMCs were isolated with Histopaque HybriMax -1077 density gradient (Sigma) from healthy human volunteers after obtaining informed consent. CD14⁺ cells were purified by positive selection using CD14⁺ microbeads in conjunction with the MiniMACS system (Miltenyi Biotech, Auborn, Calif.) by following the manufacturer's instructions. Purity, as assessed by flow cytometry, was greater than 90%.

pDC2 cells were enriched by negative selection using CD3-, CD11c- CD14-, and CD56-microbeads in conjunction with the MiniMACS system (Miltenyi Biotech, Auborn, Calif.) by following the manufacturer's instructions. The negatively enriched pDC2 cells were then enriched in the population to 5 % or greater as judged by flow cytometry using anti-CD123, HLA-DR, and CD4 antibodies (Becton Dickinson).

### Cell Stimulation with IRM Compounds

PBMC, monocytes (CD14+), pDC2-enriched, or DC1 (CD11c+ blood DC) cells were resuspended in supplemented RPMI at a concentration of 10⁶ cells/μl. 100 μl of cells (10⁵ cells) were then added to individual wells of a 96 well V-bottom plates (Nunc). Solutions containing supplemented RPMI with various concentrations of selective or non-selective compounds were prepared. Specifically, a non-selective compound resiquimod, shown below, and selective compounds I-VI were diluted to 2.2, 0.66, 0.22, 0.066, and 0.022 μM in supplemented RPMI. 100 μl of the compound dilutions were added to cells so that the final concentration of compound was 1.1, 0.33, 0.11, 0.033, and 0.011 μM, respectively. For stimulation of DC1 cells, the non-selective compound resiquimod was diluted to 64, 32, 16, 8, 4, and 2 μM. 100 μl of the compound dilutions were added to cells so that the final concentration was 32, 16, 8, 4, 2, and 1 μM. Cells were incubated at 37° C. in an atmosphere of 5% CO₂/95% air.

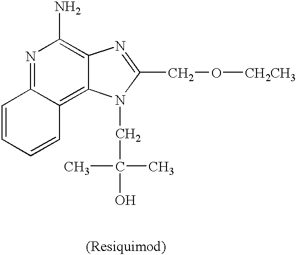

### Cell Surface and Intracellular Flow Cytometry

Evaluation of cell surface marker expression was performed by flow cytometric analysis using the following monoclonal antibodies: PE-conjugated CD14, clone MφP9, PE- and FITC-conjugated HLA-DR, clone L243, PE- and FITC-conjugated γ1/γ2a isotype control, clones X40 and X39 (all from Becton Dickinson, Mountain View, Calif.). Cells (5×10⁵) were incubated for 15 minutes incubation at 4° C. with purified IgD (Becton Dickinson) to block non-specific binding, and then the cells were stained for 30 minutes with the antibodies at 4° C. in PBS containing 10% FCS and 0.1% sodium azide. After washing in PBS, the cells were analyzed using a FACScan flow cytometer and Cell Quest software (Becton Dickinson).

### Cytokine Analysis

Cytokine levels were measured by ELISA. Human TNF-α and IL-12 (p40/p70) kits were purchased from Genzyme (Cambridge, Mass.). Human IL-6 kits were obtained from Biosource International (Camarillo, Calif.). All ELISA were run according to manufacturer's specifications. IFN levels were measured by bioassay (40). IFN-α and IFN-β specific antibodies were used to determine which type I IFN was present in the cellular supernatants. Results for all ELISAs are presented in pg/ml, whereas IFN results are presented in U/ml.

### Characterization of the Cytokine Profile by Intracellular Flow Cytometry

pDC2-containing cell populations were stimulated for 12 hours with various selective and nonselective compounds at concentrations ranging from 0.005 to 5 μM in the presence of Brefeldin A (Calbiochem) to prevent protein secretion. The pDC2-containing cell populations were then washed with PBS, fixed with 4% paraformaldehyde (Merck), and permeabilized with 0.1% saponin/PBS (Sigma). The cells were stained by using mAbs HLA-DR+-PerCP, CD123-PE (Becton Dickinson), and either TNF-FITC, IL-12-FITC or IFN-α-FITC (Chromaprobe). After washing in PBS, the cells were analyzed by three color flow cytometry using a FACScan flow cytometer and Cell Quest software (Becton Dickinson).

The data in Table 1 represents results of bioassay and ELISA of expression of cytokines IFN-α and TNF-α as measured from cell culture supernatants from various cell populations stimulated with the non-selective compound resiquimod.

The data in Table 2 represents results of bioassay and ELISA of expression of cytokines IFN-α and TNF-α as measured from cell culture supernatants from various cell populations stimulated with the selective Compound II. Stimulation with other selective Compounds I and III-VI produced similar cytokine expression levels.

TABLE 1
CYTOKINE EXPRESSION IN VARIOUS CELL TYPES STIMULATED
WITH NON-SELECTIVE COMPOUIND “RESIQUIMOD”

| | | | | |
|---|---|---|---|---|
| Resiquimod | IFN | | U IFN/10,000 | pg TNF/10,000 |
| [μm] | [U/ml] | TNF [pg/ml] | cells | cells |

PBMC

| | | | | |
|---|---|---|---|---|
| 1.1 | 4985 | 1617 | 5.0 | 1.6 |
| 0.33 | 3788 | 8 | 3.8 | 0.0 |
| 0.11 | 1263 | 0 | 1.3 | 0.0 |
| 0.033 | 3788 | 0 | 3.8 | 0.0 |
| 0.011 | 5 | 0 | 0.0 | 0.0 |

CD14 + cells

| | | | | |
|---|---|---|---|---|
| 1.1 | 140 | 2187 | 1.4 | 21.9 |
| 0.33 | 36 | 13 | 0.4 | 0.1 |
| 0.11 | 21 | 0 | 0.2 | 0.0 |

PDC2-enriched cells

| | | | | |
|---|---|---|---|---|
| 1.1 | 3788 | 1175 | 37.9 | 11.8 |
| 0.33 | 3788 | 895 | 37.9 | 9.0 |
| 0.11 | 1662 | 1293 | 16.6 | 12.4 |
| 0.033 | 2676 | 196 | 28.8 | 2.0 |
| 0.011 | 1662 | 14 | 16.6 | 0.1 |

DC11c + blood DC (DC1)

| | | | | |
|---|---|---|---|---|
| 32 | 4 | 2066 | 0.0 | 20.7 |
| 16 | 4 | 2671 | 0.0 | 26.7 |
| 8 | 4 | 3580 | 0.0 | 35.8 |
| 4 | 5 | 3828 | 0.0 | 38.3 |
| 2 | 5 | 4688 | 0.0 | 46.9 |
| 1 | 5 | 4364 | 0.0 | 43.6 |

TABLE 2
CYTOKINE EXPRESSION IN VARIOUS CELL TYPES
STIMULATED WITH SELECTIVE COMPOUND II

| | | | |
|---|---|---|---|
| IFN | | U IFN/10,000 | pg TNF/10,000 |
| [U/ml] | TNF [pg/ml] | cells | cells |

PBMC

| | | | | |
|---|---|---|---|---|
| Compound II | | | | |
| 1.1 | 4985 | 0 | 5.0 | 0.0 |
| 0.33 | 4985 | 54 | 5.0 | 0.1 |
| 0.11 | 3788 | 0 | 3.8 | 0.0 |
| 0.033 | 1 | 0 | 0.0 | 0.0 |
| 0.011 | 1 | 0 | 0.0 | 0.0 |

CD14 + cells

| | | | | |
|---|---|---|---|---|
| Resiquimod | | | | |
| [μm] | | | | |
| 1.1 | 27 | 0 | 0.3 | 0.0 |
| 0.33 | 52 | 0 | 0.6 | 0.0 |
| 0.11 | 16 | 0 | 0.2 | 0.0 |

PDC2-enriched cells

| | | | | |
|---|---|---|---|---|
| 1.1 | 6561 | 807 | 65.6 | 8.1 |
| 0.33 | 6561 | 691 | 65.6 | 6.9 |
| 0.11 | 6561 | 405 | 65.6 | 4.1 |
| 0.033 | 4985 | 54 | 49.9 | 0.5 |
| 0.011 | 140 | 1 | 1.4 | 0.0 |

We claim:

1. A method for identifying a compound that selectively induces production of IFN-α from pDC2 cells, the method comprising:
obtaining a population of cells that includes both inflammatory cytokine producing cells and pDC2 cells;
contacting the population of cells with a test compound;
determining the amount of IFN-α present in the population of cells contacted with the test compound;
determining the amount of inflammatory cytokine(s) present in the population of cells contacted with the test compound; and
identifying the test compound as a selective inducer of IFN-α if IFN-α is present in the population of cells after contact with the test compound in an amount at least three times greater than the amount of inflammatory cytokine(s) present in the population of cells.

2. The method of claim 1 wherein the amount of IFN-α and inflammatory cytokine(s) is determined from culture supernatants using ELISA or bioassay.

3. The method of claim 1 wherein the amount of IFN-α and inflammatory cytokine(s) is determined from cells in the population using a method selected from the group consisting of dot blotting, Western blotting, Northern blotting, RPA and RT-PCR.

4. The method of claim 1 wherein the inflammatory cytokine is TNF-α or IL-12.

5. The method of claim 1, wherein the population of cells is in whole blood.

6. The method of claim 1, wherein the population of cells comprises peripheral blood mononuclear cells.

7. The method of claim 1, wherein the population of cells comprises a fraction of peripheral blood mononuclear cells containing at least 5% pDC cells.

8. The method of claim 1 wherein the population of cells is contacted with the test compound at concentrations ranging from about 0.005 to 5 μM.

9. The method of claim 1 wherein the population of cells is cultured with the test compound for a period ranging from about 12 to 36 hours.

10. The method of claim 1 wherein the population of cells comprises a CD14+ cell type.

11. The method of claim 10 wherein the inflammatory cytokine is TNF-α and the amount of TNF-α produced by the CD14+ cells is undetectable when the test compound contacts the population of cells at a concentration of 1 μM.

12. A method for identifying a compound that selectively induces production of IFN-α from pDC2 cells, the method comprising:
obtaining a population of cells that includes both inflammatory cytokine producing cells and pDC2 cells;
contacting the population of cells with a test compound;
identifying pDC2 cells present in the population and determining that IFN-α is produced by the pDC2 cells by flow cytometry;
determining the production of inflammatory cytokine(s) in the population of cells by flow cytometry; and
identifying the test compound as a selective inducer of IFN-α if all cells present in the population other than pDC2 cells produce insignificant levels of inflammatory cytokine(s).

13. The method of claim 12 wherein the pDC2 cells are identified by the presence of HLA-DR and CD123 cell surface markers on a surface of the pDC2 cells.

14. The method of claim 12 wherein the inflammatory cytokine is TNF-α, IL-12 and/or IL-1.

15. The method of claim 12, wherein the population of cells is in whole blood.

16. The method of claim 12, wherein the population of cells comprises peripheral blood mononuclear cells.

17. The method of claim 12, wherein the population of cells comprises a fraction of peripheral blood mononuclear cells.

18. The method of claim 12 wherein the population of cells is contacted with the test compound at concentrations ranging from about 0.005 to 5 μM.

19. The method of claim 12 wherein the population of cells is cultured with the compound for a period ranging from about 2 to 24 hours.

20. A method of affecting a condition of a patient responsive to IFN-α, the method comprising a step of:
obtaining a population of cells that includes both inflammatory cytokine producing cells and pDC2 cells;
contacting the population of cells with a test compound;
determining the amount of IFN-α present in the population of cells contacted with the test compound;
determining the amount of inflammatory cytokine(s) present in the population of cells contacted with the test compound;
identifying the test compound as a selective inducer of IFN-α if IFN-α is present in the population of cells after contact with the test compound in an amount at least three times greater than the amount of the inflammatory cytokine(s) present in the population of cells; and
administering the identified selective compound to the patient to affect the condition.

21. The method of claim 20 wherein the amount of IFN-α and inflammatory cytokines is determined from culture supernatants using ELISA or bioassay.

22. The method of claim 20 wherein the amount of IFN-α and inflammatory cytokines is determined from cells in the population using a method selected from the group consisting of dot blotting, Western blotting, Northern blotting, RPA and RT-PCR.

23. The method of claim 20 wherein the inflammatory cytokines include TNF-α and/or IL-1.

24. The method of claim 20, wherein the population of cells is in whole blood.

25. The method of claim 20, wherein the population of cells comprises peripheral blood mononuclear cells.

26. The method of claim 20, wherein the population of cells comprises a fraction of peripheral blood mononuclear cells.

27. The method of claim 20 wherein the population of cells is contacted with the test compound at concentrations ranging from about 0.005 to 5 μM.

28. The method of claim 20 wherein the population of cells are cultured with the test compound for a period ranging from about 12 to 36 hours.

29. The method of claim 20 wherein the population of cells comprises a CD14 + or DC1 cell type.

30. The method of claim 29 wherein the inflammatory cytokine is TNF-α and the amount of TNF-α produced by the CD14 + or DC1 cells is undetectable when the test compound is in contact with the cells at a concentration of about 1 μM.

31. The method of claim 20 wherein the condition is selected from the group consisting of melanoma, myeloid leukemia, non-Hodgkin's lymphoma, renal cell carcinoma, Kaposi's sarcoma, multiple sclerosis, hypereosinophilic syndrome, myoproliferative disorders, idiopathic myelofibrosis, hepatitis B, and chronic hepatitis C, variola, influenza, parainfluenza, adenovirus, coronavirus, and rhinovirus.

32. The method of claim 31 wherein the condition is selected from the group constiting of cutaneous necrotising vasiculitis, mixed cryoglobulinemia, porphyria cutanea tarda, lichen planus, Adamantiadis-Behcet syndrome, erythema multiforme and nodosum, malacoplakia, urticaria and pruritus.

33. The method of claim 20 wherein the selective compound is delivered to a desired cell type by a targeting moiety.

34. A method for selectively inducing IFN-α production from a population of cells that includes pDC2 cells, the method comprising contacting the population of cells with an immune response modifier compound that selectively induces IFN-α production by pDC2 cells.

35. The method of claim 34 wherein the compound is Compound I.

36. The method of claim 34 wherein the compound is Compound II.

37. The method of claim 34 wherein the compound is Compound III.

38. The method of claim 34 wherein the compound is Compound IV.

39. The method of claim 34 wherein the compound is Compound V.

40. The method of claim 34 wherein the compound is Compound VI.
